(19) United States
(12) Patent Application Publication
(10) Pub. No.: US 2005/0261601 A1
(43) Pub. Date: Nov. 24, 2005

(54) Method and system for processing neuro-electrical waveform signals

(76) Inventors: Eleanor L. Schuler, Rio Rancho, NM (US); Mark Frazee, Alburquerque, NM (US); Dennis Meyer, Alburquerque, NM (US)

Correspondence Address: RALPH C. FRANCIS;FRANCIS LAW GROUP, 1942 EMBARCADERO, OAKLAND, CA, 94686, US

(21) Appl. No.: 11/147,497

(22) Filed: Jun. 7, 2005

Related U.S. Application Data
(63) Continuation-in-part of application No. 11/125,480, filed on May 9, 2005.
(63) Continuation-in-part of application No. 10/847,738, filed on May 17, 2004, now Pat. No. 6,937,903.
(60) Provisional application No. 60/578,650, filed on Jun. 10, 2004.
(60) Provisional application No. 60/471,104, filed on May 16, 2003.

(51) Int. Cl. A61B 5/04; G11C 5/00; G10H 7/00

(52) U.S. Cl. 600/545

(57) ABSTRACT

The invention comprises a processor capable of receiving, storing and processing waveform signals generated in the body and generating waveform signals that substantially correspond to waveform signals that are generated in the body and are operative in the control of a body organ function. The invention also includes a computerized system having a sensor for capturing at least one waveform signal that is generated in a subject's body and is operative in the regulation of body organ function, a processor that is capable of receiving, storing and processing the captured waveform signals and generating a waveform signal that is recognized by the body as a modulation signal, and a transmitter for delivering the generated waveform signal to the body.

### CROSS-REFERENCE TO RELATED APPLICATIONS

This application claims the benefit of U.S. Provisional Application No. 60/578,650, filed Jun. 10, 2004, and is a continuation-in-part of U.S. application Ser. No. 11/125,480, filed May 9, 2005, which in turn is a continuation-in-part of U.S. application Ser. No. 10/847,738, filed May 17, 2004, which claims the benefit of U.S. Provisional Application No. 60/471,104, filed May 16, 2003.

### FIELD OF THE PRESENT INVENTION

The present invention relates generally to medical methods and systems for the treatment and/or management of body organs and structures in humans and animals. More particularly, the invention relates to a system and method for receiving, storing, processing and generating neuro-electrical waveform signals to regulate body organ function.

### BACKGROUND OF THE INVENTION

As is well known in the art, the brain modulates (or controls) body organ function via electrical signals (i.e., action potentials or waveform signals), which are transmitted through the nervous system. The nervous system includes the central nervous system, which comprises the brain and the spinal cord, and the cranial and peripheral nervous system, which generally comprises groups of nerve cells (i.e., neurons) and peripheral nerves that lie outside the brain and spinal cord. The various nerve networks and systems are anatomically separate, but functionally interconnected.

As indicated, the nervous system is constructed of nerve cells (or neurons) and glial cells (or glia), which support the neurons. Operative neuron units that carry signals from the brain are referred to as “efferent” nerves. “Afferent” nerves are those that carry sensor or status information to the brain. Together, these components of the nervous system are responsible for the function, regulation and modulation of the body's organs, muscles, secretory glands and other physiological systems.

The typical neuron includes four morphologically defined regions: (i) cell body, (ii) dendrites, (iii) axon and (iv) presynaptic terminals. The cell body (soma) is the metabolic center of the cell. The cell body contains the nucleus, which stores the genes of the cell, and the rough and smooth endoplasmic reticulum, which synthesizes the proteins of the cell.

The nerve cell body typically also includes two types of outgrowths (or processes); the dendrites and the axon. Most neurons have several dendrites; these branch out in tree-like fashion and serve as the main apparatus for receiving signals from other nerve cells.

The axon is the main conducting unit of the neuron. The axon is capable of conveying coded electrical signals along distances that range from as short as 0.1 mm to as long as 2 m. Many axons split into several branches, thereby conveying information to different targets.

The electrical signals transmitted along the axon, referred to as action potentials (or sparks), are rapid and transient “all-or-none” nerve electrical impulses. Action potentials typically have an amplitude of approximately 100 millivolts (mV) and a duration of approximately I msec. Action potentials are conducted along the axon, without failure or distortion, at rates in the range of approximately 1-100 meters/sec. The amplitude of the action potential remains constant throughout the axon, since the impulse is continually regenerated as it traverses the axon.

A “neurosignal” is a composite signal that includes many action potentials which function as an instruction set for proper organ function. By way of example, an instruction set for the diaphragm to perform an efficient ventilation will include information regarding frequency, initial muscle tension, degree (or depth) of muscle movement, etc. Such signal transmission or application to a body can induce small breaths, large breaths, rapid or slow breathing, or pause the respiration process.

Neurosignals are thus codes that contain complete sets of information for complete organ function. These codes must be “decoded” to be understood or executed by a target organ. The present technology, described in detail herein, establishes that the neurosignals contain more accurate and complete information than previously accepted.

The prior art includes various apparatus, systems and methods that include an apparatus for or step of recording action potentials or signals, to regulate body organ function. The signals are, however, typically subjected to extensive processing and are subsequently employed to regulate a “mechanical” device or system, such as a ventilator or prosthesis. Illustrative are the systems disclosed in U.S. Pat. Nos. 6,360,740 and 6,522,926.

In U.S. Pat. No. 6,360,740, a system and method for providing respiratory assistance is disclosed. The noted method includes the step of recording “breathing signals”, which are generated in the respiratory center of a patient. The “breathing signals” are processed and employed to control a muscle stimulation apparatus or ventilator.

In U.S. Pat. No. 6,522,926, a system and method for regulating cardiovascular function is disclosed. The noted system includes a sensor adapted to record a signal indicative of a cardiovascular function. The system then generates a control signal (as a function of the recorded signal), which activates, deactivates or otherwise modulates a baroreceptor activation device.

A major drawback associated with the systems and methods disclosed in the noted patents, as well as most known systems, is that the control signals that are generated and transmitted are “user determined” and “device determinative”. Thus, the noted “control signals” are not related to or representative of the signals that are generated in the body. No attempt is made to use signals that mimic the natural neurosignals that the body uses to communicate between the organs, muscles, spinal cord and brain. Correspondingly, any signals generated by these prior art devices would not be operative in the control or modulation of a body organ function if transmitted directly thereto.

Currently available systems and methods are not designed or adapted to identify, capture, store and process neurosignals generated in the body or generate complex neurosignals at sample rates of thousands of samples per second to millions of samples per second having amplitudes of only millivolts or microvolts. Thus, the prior art has failed to fully identify and capture intact neurosignals that correspond to a given body function. The prior art has also failed to regulate body function using “generated” waveform signals that substantially correspond to neurosignals generated in the body.

It would thus be desirable to provide a processor (or computer system) that is adapted to receive or record (in real-time), store, analyze and/or process neurosignals (or waveform signals) generated in a body, and generate waveform signals that substantially correspond (or are similar) to the recorded waveform signals and are operative in the control of body organ function.

It is therefore an object of the invention to provide a processor that is adapted to receive in real-time and process neurosignals (or waveform signals) generated in the body.

It is another object of the invention to provide a processor that is adapted to categorize captured or recorded waveform signals according to the function performed by the signals.

It is another object of the invention to provide a processor that can be readily employed to operate and/or regulate at least one body organ function that includes a storage medium that is adapted to store waveform signals that are generated in the body or correspond to waveform signals generated in the body that are operative in the control of body organ function.

It is another object of the invention to provide a processor that can be readily employed to operate and/or regulate at least one body organ function that includes means for modifying waveform signals that are generated in the body.

It is yet another object of the invention to provide a processor that can be readily employed to operate and/or regulate at least one body organ function that includes means for generating waveform signals that substantially correspond to waveform signals generated in the body and are operative in the control of body organ function.

It is another object of the invention to provide a processor that can be readily employed to operate and/or regulate at least one body organ function that includes means for modifying segments of waveform signals.

It is another object of the invention to provide a processor that can be readily employed to operate and/or regulate at least one body organ function that includes means for generating a baseline signal from recorded waveform signals.

It is another object of the invention to provide a processor that can be readily employed to operate and/or regulate at least one body organ function that includes means for comparing recorded waveform signals to baseline signals.

It is another object of the invention to provide a computerized system for operating and/or regulating body organ functions that includes means for recording waveform signals that are generated in the body, a processor adapted to receive, record and/or store, analyze and/or process the received waveform signals, and generating waveform signals that substantially correspond to the recorded waveform signals and are operative in the control of body organ function, and means for transmitting generated waveform signals to the body.

It is another object of the invention to provide a computerized system for regulating body organ function that can be readily employed in the assessment and/or treatment of multiple disorders, including, but not limited to, sleep apnea, respiratory distress, asthma, acute low blood pressure, abnormal heart beat, paralysis, spinal chord injuries, acid reflux, obesity, erectile dysfunction, a stroke, tension headaches, a weakened immune system, irritable bowel syndrome, low sperm count, sexual unresponsiveness, muscle cramps, insomnia, incontinence, constipation, nausea, spasticity, dry eyes syndrome, dry mouth syndrome, depression, epilepsy, low levels of growth hormone and insulin, abnormal levels of thyroid hormone, melatonin, adrenocorticotropic hormone, ADH, parathyroid hormone, epinephrine, glucagon and sex hormones, pain block and/or abatement, physical therapy and deep tissue injury.

### SUMMARY OF THE INVENTION

In accordance with the above objects and those that will be mentioned and will become apparent below, the processor (hereinafter “neurocomputer”) of the invention for regulating body organ function generally includes means for receiving waveform signals that are generated in the body, a storage medium for storing received waveform signals, means for processing stored waveform signals and means for generating waveform signals that substantially correspond to the received waveform signals and are operative in the control of at least one body organ function.

In one embodiment of the invention, the means for receiving waveform signals is adapted to receive signals having a rate of at least approximately 10,000 S/s, more preferably, at least approximately 1 MS/sec. (samples per second)

In one embodiment, the storage medium stores the received (or recorded) waveform signals categorized by specific body organ function.

In one embodiment, the means for processing stored waveform signals modifies the stored waveform signal. Preferably, the means for processing stored waveform signals modifies the stored waveform signal by adjusting a characteristic selected from the group consisting of frequency, voltage, pacing (or bursting) and amperage.

In another embodiment, the means for processing stored waveform signals frequency modulates the stored waveform signal. In one aspect, the signals are modulated in the range of approximately 450-550 Hz, more preferably, approximately 500 Hz.

The noted frequency, i.e., approx. 500 Hz, has been found to be the best frequency for penetrating the myelination of a nerve. As will be appreciated by one having ordinary skill in the art, the frequency can be varied to accommodate a desired target organ (e.g., muscle) and/or body composition (e.g., fat, skin, etc.).

In a further embodiment of the invention, the means for processing stored waveform signals modifies a segment of the stored waveform signal by copying, cutting, pasting, deleting, cropping, appending, building or inserting segments of stored waveform signals.

In one embodiment of the invention, the means for generating a waveform signal is adapted to provide signals at a rate of at least approximately 1 Mbps (bits per second), more preferably, at least approximately 5 Mbps.

In a further embodiment, the integrated, computerized system (hereinafter “neurocode system”) of the invention for recording, storing, analyzing, processing, generating and transmitting waveform signals to regulate body organ function generally includes a sensor for capturing at least a first waveform signal that is generated in a subject's body and is operative in the regulation of body organ function, a neurocomputer adapted to receive the captured waveform signal, store the captured waveform signal, analyze and/or process the stored waveform signal and generate at least a second waveform signal that substantially corresponds to the first waveform signal and is recognizable by at least one body organ as a modulation (or operational) signal, and a transmitter for delivering the second waveform signal to the body.

In one embodiment, the first waveform signal captured by the sensor is converted from analog form to digital form.

In one embodiment of the invention, the sensor is adapted to provide direct connection to a nerve in the subject.

In one embodiment, the neurocomputer modifies the stored waveform signal by converting the waveform signal from digital form to analog form.

In another embodiment, the neurocomputer modifies the stored waveform signal by adjusting the frequency, voltage or pacing of the signal.

In one embodiment of the invention, the sensor comprises a high speed sensor. Preferably, the sensor has a sample rate of at least approximately 250,000 S/sec., more preferably, up to approximately 1 MS/sec.

In one embodiment of the invention, the transmitter is adapted to provide direct connection to a nerve in the subject. Alternatively, the transmitter is adapted to provide indirect communication with a subject's body, preferably using magnetic, electromagnetic, ultrasonic, sonic, seismic and/or broadband means.

In a further embodiment of the invention, the neurocode system includes a low speed sensor. The noted sensor can include a respirator, a pneumotach, a pulse rate monitor, an airflow monitor, a vitals monitor, a temperature sensor, a motion sensor and a pressure sensor.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a schematic illustration of one embodiment of a neurocode system, according to the invention;

FIG. 2 is a schematic illustration of one embodiment of a storage medium, according to the invention;

FIGS. 3A-3B and 4A-4B are illustrations of waveform signals that are operative in the control of the respiratory system, which were captured from the phrenic nerve of a subject by the neurocode system of the invention; and

FIGS. 5A and 5B are illustrations of waveform signals that were modified by the computerized system of the invention.

### DETAILED DESCRIPTION OF THE INVENTION

Before describing the present invention in detail, it is to be understood that this invention is not limited to particularly exemplified apparatus, systems, structures or methods as such may, of course, vary. Thus, although a number of apparatus, systems and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “a waveform signal” includes two or more such signals; reference to “a neuron” includes two or more such neurons and the like.

### DEFINITIONS

The term “nervous system”, as used herein, means and includes the central nervous system, including the spinal cord, cranial nerves, medulla, pons, cerebellum, midbrain, diencephalon and cerebral hemisphere, and the peripheral nervous system, including the neurons and glia.

The terms “waveform” and “waveform signal”, as used herein, mean and include a composite electrical signal that can be generated in the body and carried by nerves (or neurons) in the body, including neurocodes and components and segments thereof.

The term “body organ”, as used herein, means and includes, without limitation, the brain, cranial nerves, skin, bones, cartilage, tendons, ligaments, skeletal muscles, smooth muscles, heart, blood vessels, brain, spinal cord, peripheral nerves, nose, eyes, ears, mouth, tongue, pharynx, larynx, trachea, bronchus, lungs, esophagus, stomach, liver, pancreas, gall bladder, small intestines, large intestines, rectum, anus, kidneys, ureter, bladder, urethra, hypothalamus, pituitary, thyroid, adrenal glands, parathyroid, pineal gland, ovaries, oviducts, uterus, vagina, mammary glands, testes, seminal vesicles, prostate, penis, lymph nodes, spleen, thymus and bone marrow.

The terms “patient” and “subject”, as used herein, mean and include humans and animals.

The term “plexus”, as used herein, means and includes a branching or tangle of nerve fibers outside the central nervous system.

The term “ganglion”, as used herein, means and includes a group or groups of nerve cell bodies located outside the central nervous system.

The terms “processor” and “neurocomputer”, as used herein, mean and include a digital computing device adapted to receive, store, process and generate waveform signals that are generated by the body or substantially correspond to neurosignals generated by the body.

The present invention substantially reduces or eliminates the disadvantages and drawbacks associated with prior art methods and systems for regulating body organ function. As discussed in detail herein, the invention exploits the ability to replicate the exact nerve signals, i.e., neurosignals (referred to herein as “waveform signals”), that have been isolated and captured (or recorded) from the brain or other parts of the nervous system or signals that substantially correspond to the recorded signals. The noted signals can be employed for use as or in conjunction with medical treatment, medical diagnosis, medical research, etc. By using waveform signals that correspond to natural neurosignals, the methods and systems of the invention are able to operate at approximately one volt or less.

In one embodiment, the invention thus comprises a neurocomputer that is adapted to receive, store, analyze and/or process neurosignals or waveform signals generated in the body of a subject (human or animal), and generate waveform signals corresponding to the neurosignals, allowing the generated signals to be broadcast, transmitted or conducted into appropriate areas of a subject's body to cause operation, adjustment, regulation or manipulation of target organs, and glandular or muscle systems.

According to the invention, the generated nerve-specific waveform instruction (i.e., waveform signal(s)) can be employed to, for example, restore breathing, restart hearts, eliminate pain, reduce or raise blood pressure, restore sexual function, regulate bladder and bowel functions, reduce weight, move appendages, such as legs and arms, and wet dry eyes, via implants or transdermally, without harmful additional voltage or current.

Generally, the neurocomputer of the invention is adapted to receive waveform signals at sufficiently high sample rates to maintain the signal integrity necessary for the signal to control a body organ function. The neurocomputer is also adapted to store waveforms, preferably categorized by the body organ function controlled by the waveforms.

The neurocomputer is also adapted to analyze and process the stored waveform signals. According to the invention, processing of the signals includes retrieving the stored waveform signals from a storage medium and optionally modifying the signals to alter or modulate the function coded in the waveform signals or to optimize the waveform signals for transmission to the body. Processing can also include comparing a plurality of waveform signals received from one or more subjects to aid in identifying specific patterns or control functions.

Processing the signals can additionally include modifying or editing waveform signals to effectuate one or more signal bursts and/or silencing, delaying and sustaining one or more signals.

Processing the signals can further include modifying or editing waveform signals by copying, cutting, pasting, deleting, cropping, appending or inserting desired segments of waveform signals.

Additionally, the neurocomputer is preferably adapted to generate waveform signals for transmission to the body, wherein the generated waveform signals have a sample rate sufficient to be recognized by the desired (or target) body organ as a neurosignal operative in the control of that body organ. The generated waveform signals also preferably have the capability to travel on or within the nerve structures that lead to the target body organ.

In one embodiment of the invention, the neurocomputer of the invention is integrated into a computerized “neurocode” system that is adapted to isolate, capture and record (in real-time), store, analyze and/or process waveform signals generated in the body, and generate and transmit waveform signals to a subject's body to regulate body organ function. Preferably, the neurocode system includes a sensor adapted to capture at least one waveform signal that is generated in a subject's body and is operative in the regulation of body organ function, a neurocomputer (as described above) that is adapted to generate at least one waveform signal that substantially corresponds to the captured waveform signal and is recognizable by at least one body organ as a modulation signal, and a transmitter for delivering the generated waveform signal to the body.

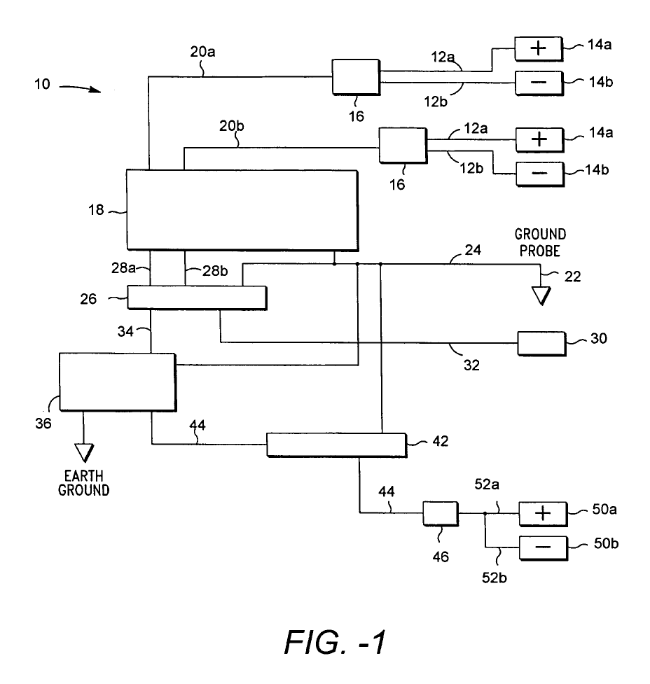

Referring now to FIG. 1, there is shown one embodiment of a neurocode system 10 for regulating body organ function. As illustrated in FIG. 1, the electrical leads 12a and 12b of the positive and negative “high speed” signal probes 14a and 14b, respectively, are preferably connected to a high impedance head-stage or isolation preamplifier 16. As will be appreciated as one having ordinary skill in the art, various pre-amps can be employed within the scope of the invention. In a preferred embodiment of the invention, the pre-amp 16 comprises a Super-Z high-impedance preamplifier manufactured by CWE, Inc.

As is known in the art, the noted preamplifier has a very high impedance, low drift, differential input amplifier and a built in DC off-set adjustment. The use of a high-impedance preamplifier helps ensure that electrical power from the system is isolated from the subject. The unit is preferably set to the AC (alternating current) mode, which eliminates any DC (direct current) off-sets. The amplifications of the unit are also preferably set to 1.0. In this embodiment, the preamplifiers have an output capability in the range of approximately 0-10 V and 0-10 mA.

As illustrated in FIG. 1, the signal is routed from the high impedance head-stage preamplifier 16 to the bioamplifier 18 via leads 20a and 20b. The ground probe 22 is also in communication with the bioamplifier 18 via lead 24. In one embodiment, the bioamplifier 18 is preferably set to magnify the waveform signal 50-fold to produce a desirable signal.

As will be appreciated by one skilled in the art, the captured nerve signal(s) will include the waveform signal representative of the signal produced in the body as well as background noise and extraneous material. Thus, bioamplifier 18 preferably filters the captured signal to substantially reduce, more preferably, eliminate, the background noise and extraneous material.

According to the invention, various conventional apparatus and techniques can be employed to filter the captured signals. In a preferred embodiment, the bioamplifier 18 incorporates a 4 pole Butterworth filter with resultant attenuation of −12 dB/octave for frequencies outside of the selected cutoff frequencies signals to filter the signals.

Further, bioamplifier 18 incorporates cutoff filters to reduce signal noise, such as noise generated by AC powered 60 Hz electrical equipment. The noted filters include a high frequency cutoff filter operating in the range of approximately 100 Hz to 50 kHz, preferably at approximately 10 kHz, and a low frequency cutoff filter operating in the range of approximately 1 Hz to 300 Hz, preferably at approximately 1 Hz. Generally, the cutoff filters eliminate all signals having a frequency outside the limit.

In addition to filtering the captured signals, bioamplifier 18 also amplifies the signal, preferably in increments in the range of approximately 50 to 50,000. Bioamplifier 18 preferably amplifies both AC and DC signals, while causing little or no distortion in the passed signal.

In one embodiment of the invention, the amplified signal from bioamplifier 18 is transmitted (or routed) to the analog to digital conversion unit 26 via leads 28a and 28b, which is adapted to convert the signal from an analog format to a digital format. This conversion makes the waveform signal easy for the neurocode system 10 to display, read, process and store by changing the analog wave of information into a stream of digital data points. As will be appreciated by those having skill in the art, translating the waveform signal from analog to digital format allows for computer based analysis, digital copying and transmission, and repeatable play-back.

According to the invention, various analog to digital converters can be employed to provide the noted conversion. In a preferred embodiment of the invention, the conversion apparatus comprises a National Instruments Corporation FireWire data acquisition card (Part number DAQ Pad 6070E).

Referring to FIG. 1, a further embodiment of the invention utilizes a low speed input probe 30 to capture a biological signal. The signal captured by the low speed probe 30 is routed directly to the analog to digital converter 26 via lead 32 and subsequently digitized. The ground probe 22 is similarly routed to the analog to digital converter 26 via lead 24.

The biological signal can correspond to a number of conditions that are sensed using low speed probe 30 according to the invention. In one embodiment, probe 30 is adapted to monitor lung tidal volume. In the noted embodiment, probe 30 translates the positive and negative pressures involved in breathing into electrical signals. Suitable input pressure transducers are capable of handling the anticipated volume of the subject's lung smallest mammals to the largest. As such, a preferred input transducer is capable of measuring tidal volumes in the range of approximately 0.1 ml to 1000 ml and converting the tidal volumes into electrical signals.

In alternate embodiments, sensor information from a respirator, a pneumotach, a pulse rate monitor, an airflow monitor, a vitals monitor, or other medical device can be employed. Examples of suitable probes thus also include temperature sensors, motion sensors and pressure sensors.

According to the invention, the analog to digital converter 26 is preferably capable of handling up to 8 separate high speed analog input differential channels at sampling rates of at least 10,000 S/sec., more preferably, up to approximately 1 MS/sec. In one embodiment, up to 8 separate digital output channels are controlled. The analog to digital converter 26 further includes a timing trigger to control the rate of sampling, which is preferably in the range of approximately 10 kHz to 20 MHz.

In order for the neurocode system 10 of the invention to be useful for medical treatment, each waveform signal must be identified and characterized as to its specific purpose relating to body homeostasis or function. Accordingly, the digital signals from analog digital converter 26 are routed by cable 34 to the neurocomputer 36 of the invention. As will be appreciated by one having skill in the art, the neurocomputer 36 can include various operating systems. In a preferred embodiment, the neurocomputer 36 includes a Windows® operating system.

As discussed above, neurocomputer 36 is adapted to receive waveform signals generated in the body (in real-time), store and process the captured waveform signals, and generate at least one, preferably, a plurality of waveform signals at data rates sufficient to retain the signals' ability to modulate body organ function. To that end, neurocomputer 36 preferably operates at speeds of at least approximately 1.5 GHz or higher.

Neurocomputer 36 is also able to process waveform signals having a sample rate of at least 10,000 S/sec., more preferably, up to approximately 1 MS/sec.

Preferably, neurocomputer 36 is adapted to communicate with each component (or sub-system) of neurocode system 10 at data rates of at least approximately 1 Mbps, more preferably, up to at least approximately 5 Mbps.

Further, neurocomputer 36 is adapted to receive waveform signals having voltages in the range of approximately −10 to +10 V.

Preferably, the neurocomputer 36 can generate waveform signals at a rate of at least 10,000 S/sec., more preferably, at least 3 MS/s, even more preferably, up to approximately 5 MS/sec.

In preferred embodiments of the invention, the nominal output voltage of the generated waveform signals is in the range of approximately 1 to 2 V. Also preferably, the adjusted signals do not exceed 0.25 A.

Preferably, AC voltage signals with no DC offset do not exceed the level that damages muscle tissue. The noted AC voltage is thus preferably maintained in the range of approximately 1.0-100 V.

Similarly, DC voltage signals do not exceed the level that damages the nerves. The noted DC voltage is thus preferably maintained in the range of approximately 0.0-3.0 V.

According to the invention, the neurocomputer can provide generated waveform signals having a variance (i.e., accuracy) of approximately ±0.01 mV.

Referring back to FIG. 1, in one embodiment of the invention, the neurocomputer 36 is preferably adapted to store and display the high speed digitized signals from probes 14a and 14b and the low speed digitized signal from probe 22. As desired, neurocomputer 36 stores captured waveform signals, analyzes and modifies the signals (if necessary or desired), compares captured or modified signals, generates waveform signals and displays captured and/or generated waveform signals.

As discussed above, the neurocomputer 36 is adapted to process the waveform signals. In one embodiment, processing comprises retrieving a desired waveform signal from storage. In further embodiments, processing the waveform signal comprises modifying the waveform signal.

In one embodiment of the invention, modification of the captured waveform signal includes changing the waveform signal into a positive voltage only signal. In another embodiment, modification comprises creating an envelope of the waveform signal and placing frequency modulation within that envelope.

According to the invention, modification of the captured waveform signal also includes adjusting the frequency, voltage or pacing prior to rebroadcast of the waveform signal into a subject. Preferably, the signal is adjusted, amplified or attenuated to compensate for resistance encountered during a medical treatment process and configured to avoid damage to the nerves, muscles or organs.

In additional envisioned embodiments of the invention, the neurocomputer 36 is adapted to process the waveform signal by performing analysis algorithms on the waveform signal to classify the body function controlled by the signal. In additional embodiments, the neurocomputer 36 is also adapted to process the waveform signal to correct or alter a recorded signal to provide a desired function of the bodily system controlled by the signal.

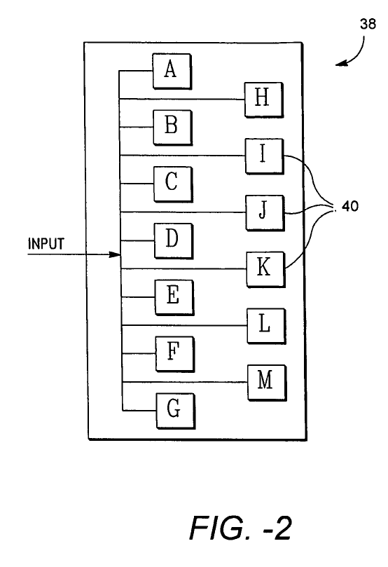

Referring now to FIG. 2, there is shown one embodiment of a storage module 38 of neurocomputer 36. As illustrated in FIG. 2, the storage module 38 includes a plurality of cells 40 (or files) that are adapted to receive at least one captured signal that is operative in the control of a target organ or muscle. By way of example, storage cell A can comprise captured signals operative in the control of the respiratory system; storage cell B can comprise captured signals operative in the control of the cardiovascular system, etc.

Preferably, the neurocomputer (or programming means thereof) of the invention is further adapted to store the captured signals according to the function performed by the signal. According to the invention, the noted signals can be stored separately within a designated storage cell 40 (e.g., storage cell A) or in a separate sub-cell.

According to the invention, the stored signals of each cell (e.g., A) and/or sub-cell can subsequently be employed to establish a base-line signal for each body function or organ. The neurocomputer 36 can then be programmed to receive a plurality of signals from one or more probes, compare the signals to the baseline signals to identify specific signals and store the identified signals in the appropriate cell 40.

In further envisioned embodiments of the invention as discussed above, the neurocomputer 36 is further programmed to compare “abnormal” signals captured from a subject and generate a modified base-line signal for transmission back to the subject. Such modification can include, for example, increasing the amplitude of a respiratory signal, increasing the rate of the signals, etc.

As discussed above, the neurocode system 10 is adapted to isolate, capture (or record) and store digitized waveform signals operative in the regulation of vegetative body organs, glandular systems, muscle systems and selected brain structures. The neurocode system 10 further includes means for outputting individual coded regulatory waveform signals.

Referring back to FIG. 1, access to a desired waveform signal for transmission to a subject is preferably obtained from storage module 38. At a minimum, the desired signal is retrieved from memory. According to one embodiment of the invention, the neurocomputer 36 generates a waveform signal by routing the digital representation of the selected waveform signal retrieved from memory to the digital to analog converter 42 via lead 44 to convert the signals to an analog format. As discussed above, the retrieved signal can be an unmodified signal recorded from the body or a signal that has been modified.

According to the invention, various digital to analog converters can be employed within the scope of the invention to provide the desired conversion. In a preferred embodiment, the converter 42 comprises a National Instruments DAQ Pad-6070E converter.

The digital to analog converter 42 similarly preferably accommodates at least 10,000 S/sec., more preferably, up to approximately 1.0 S/sec. The digital to analog converter 42 is also capable of generating at least two separate analog output channels and includes a timing trigger function, as discussed above.

In one embodiment, neurocode system 10 includes 8 high speed input channels; two low speed input channels and two high speed output channels. One having ordinary skill in the art will readily recognize that the number and type of channels is easily changed to match what is required for capturing or transmission of signals.

Following conversion from digital to analog, in one embodiment, the waveform signal is routed from the digital to analog converter 42 to a signal conditioner, such as a biphasic (or monophasic) stimulus isolator 46, via lead 48. The isolator unit 46 is adapted to isolate the signal sent to the subject from the rest of the electronics.

The biphasic stimulus isolator 46 is preferably set to provide a constant current throughout the waveform signal. In a preferred embodiment, the varying voltages are preferably converted to percentages of ±10 V throughout the signal.

By way of example, if a specific point in the analog waveform signal equals 6 V, then the percentage equals 60%. This percentage, i.e., 60%, is then used to calculate the current to be sent out. If the isolator 260 is set to an output range of 10 mA, then 60% results in 6 mA of output at that point in the analog waveform.

As the voltage of the analog waveform signal changes from zero to the maximum peak, the output from the isolator 46 will preferably have varying levels of current from zero to the corresponding percentage of the output range. The isolator 46 will thus ensure that the current being supplied is constant regardless of the changing resistance of the body.

In one embodiment of the invention, an oscilloscope is used to display the waveform signal transmitted from the isolator 46. The waveform signal shape should match what was displayed on the output window's graph. Indeed, the only possible change should be the amplitude or voltage of the waveform signals coming from the isolator 46.

Preferably, the signal conditioner can accommodate up to 8 separate analog or digital input or output differential channels at sampling rates up to approximately 1 MHz. The signal conditioner is capable of receiving the timing trigger discussed above in order to synchronize inputs and outputs.

In a preferred embodiment, data transmissions between the neurocomputer 36, the analog to digital converter 26, the digital to analog converter 42 and the biphasic isolator 46 are up to 5 Mbps. Also preferably, the neurocomputer 36 has a storage capacity of 10 GB or more to ensure that the system 10 can properly handle the display of the signals on a monitor, and can handle the acquisition and transmission of data at full speed without errors.

As illustrated in FIG. 1, in one embodiment, the waveform signal transmitted from the biphasic stimulus isolator 46 is routed to probes 50a and 50b by leads 52a and 52b, respectively.

As will be appreciated by one having ordinary skill in the art, it is also possible to develop a digital to analog conversion unit, which would provide enough electrical power to eliminate the need of the isolator 46. Care would, however, need to be exercised to ensure that this modified digital to analog conversion unit could also perform the function of isolating the body from the rest of the electronics.

In alternative embodiments of the invention, the analog to digital and digital to analog converters 26 and 42 are eliminated. This is achieved by employing a pulse rate detector for input sampling and a pulse rate generator for output signal generation. The threshold for detection of pulses and the amplitude of generated pulses will be readily observed to be a direct function of the size of the nerve and the contact area of the electrodes employed.

In alternative embodiments, the functions described in the existing preferred embodiment of a neurocomputer may be performed by utilizing discreet logic circuits, programmable logic arrays, microprocessors or microcontrollers, or Application Specific Integrated Circuits designed for the nerve detection and stimulus generation.

Various apparatus and methods have been described in the art and employed to capture waveform signals from the body. The conventional apparatus and methods typically communicate with the nerves via direct attachment of the apparatus (e.g., probe) to a target nerve. Such contact can be by a tungsten, silver, copper, platinum or gold wire. In addition, electrodes constructed of composite metals can be introduced for the direct nerve connection systems. Illustrative are the probes manufactured by World Precision Instruments, and Harvard Apparatus, sold under the trade names Metal Electrodes Tungsten Profile B and Reusable Probe Point 28 gauge 9.5 mm length, respectively.

Other suitable probe designs are discussed in co-pending U.S. patent application Ser. No. 11/125,480, filed May 9, 2005, which is hereby incorporated by reference in its entirety.

Generally, direct electrical contact input electrical probes are of different sizes in order to firmly communication with or attach to nerves without damage from a nerve diameter of 0.2-6.5 mm. Preferably, the noted probes grasp, pinch, wrap around or otherwise engage nerves with non-destructive mechanisms.

A key feature of the present invention is that the signals generated and transmitted to a subject by the neurocode system 10 are representative of the neurosignals (or waveform signals) generated in the body. More particularly, the waveform signal(s) transmitted to the subject substantially correspond to at least one waveform signal generated by the body and are operative in the control of at least one body organ (i.e., recognized by the brain or a selected organ as a modulation or control signal). The waveform signal performs the actual communication or signaling by firing neurons in patterns that cause obedient response by organs, glands, muscles, or the brain structures.

According to the invention, the waveform signals generated by the neurocomputer 36 (or processing means thereof) can be transmitted (or broadcast) to a subject by various conventional means (discussed in detail below). In a preferred embodiment, the signals are transmitted to the nervous system of the subject by direct conduction, i.e., direct engagement of a signal probe (or probes) to a target nerve. For direct connection, probes suitable for the recording of waveform signals as discussed above can be used. For implanted probes, the electrodes are preferably biocompatible, either being formed from suitable biocompatible metals or non-metals, or being coated with insulative and non-reactive substances like Mylar or Teflon to resist corrosive attack by the body and to serve as insulators where required.

For small nerves (e.g., rat nerves), the hook probes are preferably still employed (with the signal probe cradling the target nerve and the ground probe attached to an interior muscle). The surgeon must, however, exercise extreme care when isolating the target nerve. The target nerve cannot be frayed, stretched too much, or twisted. Even slight damage will diminish the effect of the transmitted waveform signal.

For larger nerves (e.g., dog, pig, human), there are a variety of nerve probes that can be employed to transmit the signal(s) to the subject. By way of example, needle probes (e.g., World Precision Instruments PTM23B05) can be inserted into the target nerve. Nerve cuffs or spiral cuffs, which wrap around nerve forcing the electrodes to make contact with the target nerve, can also be employed.

In alternative embodiments of the invention, the signals are transmitted externally via a signal probe (or probes) that is adapted to be in communication with the body (e.g., in contact with the body) and disposed proximate to a target nerve or selected organ.

For example, magnetic stimulation of nerves is possible (e.g., Magstim Magstim 200). Transcutaneous electrical nerve stimulators (TENS) units, e.g., Bio Medical BioMed 2000, which magnetically stimulate the nerve through the skin, can also be employed. A laser can also be employed to stimulate the target nerve; or electromagnetic stimulation may be employed. Finally, ultrasonic, sonic, seismic, broadband and/or other, non-invasive transmission of the signals is also possible, using microphones, seismic sensors, photonics, laser, other electromagnetic device or any combination thereof, wherein the signal is captured by a receiving antenna that is in communication with a target nerve.

According to the invention, delivery of the waveform signal to the subject is not based upon a particular probe or probe design. Thus, a user can select a specific probe for a specific procedure.

Further, the transmitted signal can be transmitted to virtually any target nerve in the nervous system. Preferably, the signal is transmitted to a branch of the effector nerve proximal to divisional ganglia, which branch to various portions of the target muscle or organ. In the case of the phrenic nerve, a preferred location is between the plexi in the neck and the diaphragm.

As is known in the art, the parameters for stimulating a nerve will change from nerve to nerve, organ to organ, and from human to human and animal to animal. Applicants have found that a DC (direct current) voltage of over 2.5 V can damage the phrenic nerve and an AC (alternating current) voltage of over 5 V can contract the diaphragm muscle too much and cause pain and/or damage.

For proper stimulation of the target nerve of a human, in accordance with the invention, the amount of voltage of the waveform signal is thus preferably set to a low value. Preferably, the maximum transmitted voltage is in the range of 100 mV-50 V, more preferably, in the range of 100 mV-5.0 V, even more preferably, in the range of approximately 100-500 mV (peak AC). In a preferred embodiment, the maximum transmitted voltage is less than 2 V.

Preferably, the amperage is less than 2 A, more preferably, in the range of 1 μA-24 mA, even more preferably, in the range of 1-1000 μA. In a preferred embodiment, the amperage is in the range of 1-100 μA.

As discussed above, modification of the signal can improve transmission of the generated waveform signal to the target area of the subject's body. Indeed, as will be appreciated by one having ordinary skill in the art, the signal often must pierce skin, fur, muscle, fat layers (lipids) and myelin sheaths, all of which serve as natural insulators. Thus, according to the invention, frequency modulation can be tailored to facilitate effective transmission of the signal through fat layers and connective tissue, as well as the myelination of the nerve.

In a preferred embodiment of the invention, the neurocomputer 36 is equipped with software that is adapted to record, store, analyze and/or process recorded waveform signals and generate organ specific waveform signals. The software is thus adapted to perform the necessary functions to control the hardware discussed above.

In a preferred embodiment, the software is designed and adapted to at least configure the different channels (high and low speed input and high speed output), display waveform signals and/or bodily function signals, record and store waveform signals and/or bodily function signals in memory, generate waveform signals at different magnifications and at varied rates, compare different waveform signals and/or bodily function signals, capture segments of waveform signals and/or bodily function signals for isolating key segments of signals, modify or convert waveform signals into electrically positive signals, modify waveform signals into an envelope of the signal, place a frequency modulation within the envelope of the waveform signals, and/or allow for manual creation of waveform signals by mapping key points of a captured waveform signal.

In one embodiment of the invention, the software configures the input channels and/or output channels to perform a desired function. As indicated above, input channels are divided into high speed and low speed channels. In the noted embodiment, the user can set the sampling rate of the high speed channels through the software individually or in groups, in the range of approximately 10 kHz to 1 MHz. Also preferably, the software permits adjustment of the input range for the high speed channels in the range of approximately 1 mV-10 V.

Further, the software allows for selection among multiple hardware devices for obtaining the high speed inputs. Similarly, the user can set the sampling rate and input range and select among low speed devices.

The software can also be used to select the acquisition duration, which is displayed on the neurocomputer screen, for the high speed and low speed input channels. In one embodiment, the acquisition duration is preferably selectable in the range of approximately 0.01-10 sec.

In the noted embodiment, the software preferably includes a manually selected scaler value function to allow for easy conversion (e.g., millimeters of mercury per second into cubic centimeters per second). The software also provides for the setting of an offset for any given low speed channel to compensate for the default range of a given device (e.g., if a device's at-rest input to the software is 3 V then the offset would be set to −3 V).

In one embodiment, the software regulates the selection and output of a desired waveform signal. A desired waveform signal can be selected from an appropriate file in the computer's memory and viewed. The output device can also be selected from a list of available hardware output devices.

Preferably, the software provides control of any modifications that may be made to the desired waveform signal. For example, the output scale factor by which the waveform signal is magnified or reduced can be selected through the software. Preferably, the output scale factor is selectable in the range of approximately 0.01 to 100.

According to the invention, the software is also adapted to effectuate switching of the analog to digital and digital to analog converters 26, 42, employed in the neurocode system 10 discussed above.

Further, as discussed above, the waveform signal can be frequency modulated. In this embodiment, the software provides the capability to mirror the envelope file, place a frequency modulation within the envelope, and magnify the frequency modulated signal, as needed.

In a preferred embodiment, the software provides single trigger and multiple trigger options. A single trigger transmits the waveform signal once per manual activation. A multiple trigger transmits the waveform signal at the rate selected by the output interval. The results from the selected input channels can also be displayed at the same rate. The output interval can be selected through software, for example, in the range of approximately 0.01-1 sec.

The software also preferably provides a variety of options for viewing the recorded and transmitted signals, and the high and low speed input and high speed output channels. Preferably, the viewing option is configured to coordinate with the recording functions, wherein the incoming signal is displayed and recorded when selected.

In one embodiment, the software displays or records any desired combination of the high and low speed input channels. Preferably, this allows correlation of a signal from the low speed input channel with the waveform signal captured on a high speed input channel.

In a further embodiment, the software provides a clipping function, wherein a segment of a recorded waveform signal is selected. According to the invention, any number of segments of a recorded signal can be selected. The segments can also be modified, as desired. For example, negative electrical portions of the segment can be eliminated.

One or more segments of a recorded waveform signal can also be edited by copying, cutting, pasting, deleting, cropping, inserting and appending the segments which each other, to create new waveform signals based upon the recorded segments.

Preferably, the software is configured to perform the noted operations using a graphical interface to allow the user to visualize the segments of the waveforms being edited.

According to the invention, the software can also provide other analytical tools. For example, the volume of a selected segment of a waveform signal can be calculated.

Another tool that is preferably provided by the software is the ability to over plot waveform signals. Any desired number of waveform signals can thus be displayed on a single field and moved or modulated with respect to each other. As can be appreciated, the noted function allows patterns within the waveform signals to be compared and analyzed.

Preferably, the software is a text and icon-based application, which employs a graphical user interface that controls the functions described above. As one having ordinary skill in the art will recognize, the above features can developed using stand-alone executables and shared libraries.

### EXAMPLES

The following examples are provided to enable those skilled in the art to more clearly understand and practice the present invention. The examples should not be considered as limiting the scope of the invention, but merely as being illustrative thereof.

### Example 1

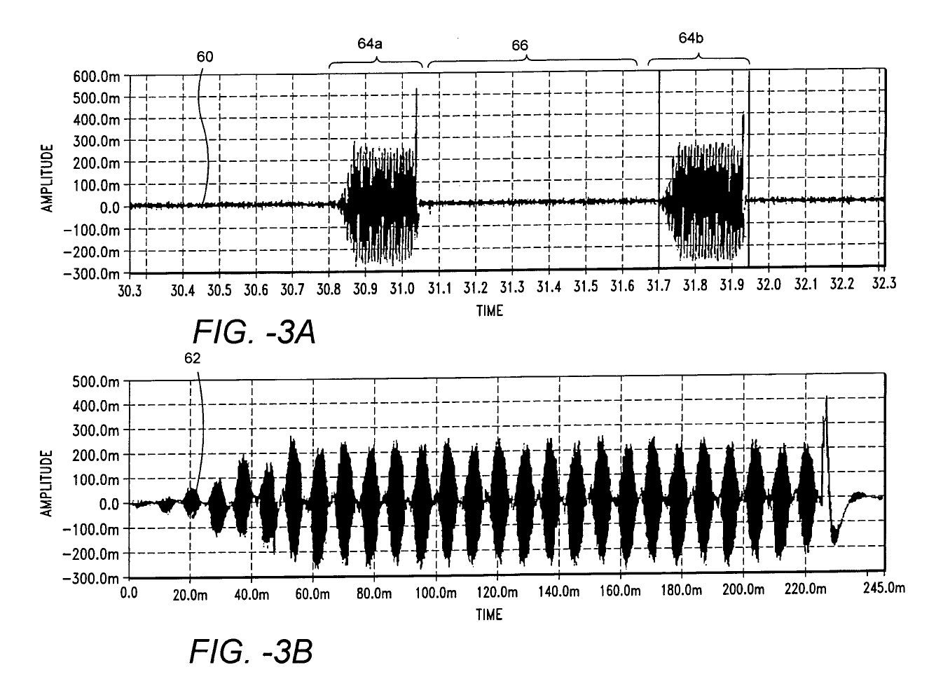

Referring now to FIGS. 3A-3B, there are shown traces 60 and 62 having waveform signals 64a and 64b that were acquired by the neurocode system of the invention. The signals 64a and 64b, which are operative in the control of the respiratory system, were captured from the phrenic nerve.

FIG. 3A shows the two signals 64a and 64b, having a rest period 66 therebetween. FIG. 3B shows an expanded view of signal 64b.

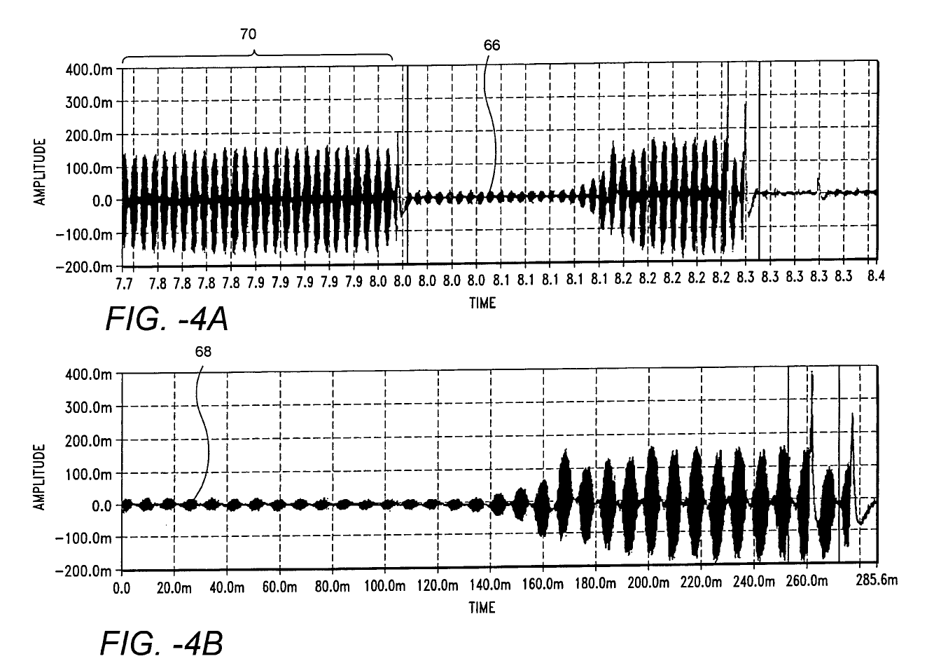

Referring now to FIGS. 4A-4B, there are shown signals 66 and 68, which were similarly acquired by the neurocode system of the invention. The noted signals 66, 68 reflect a rat in distress (i.e., going into shock). In comparison to FIG. 3A, it can be seen that the pattern of the signal 66 has changed greatly as the rat tries to breathe rapidly. In segment 70 of signal 66 it can be seen that the initial segment is longer and the number of pulses is greater.

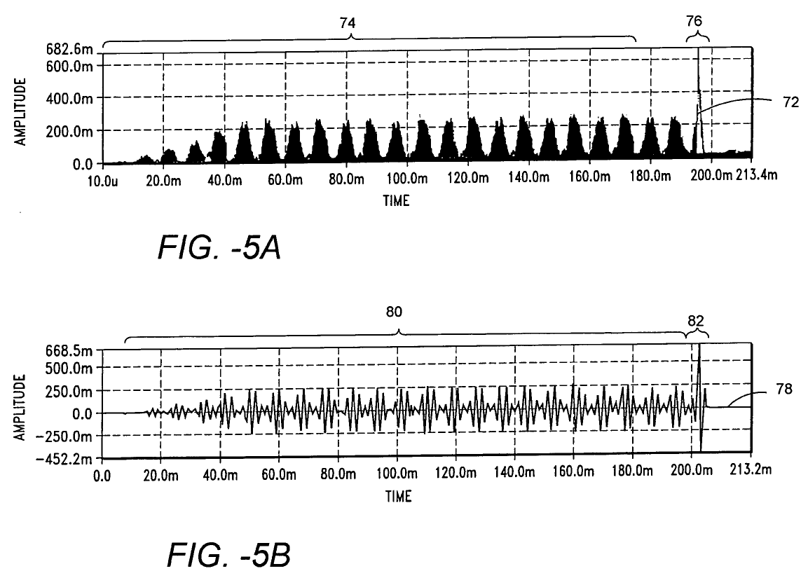

Referring now to FIGS. 5A and 5B, there are shown signals 72 and 78, which substantially correspond to waveform signals generated in the body that were processed and generated by the neurocomputer of the invention. The noted signals are merely representative of the signals that can be generated by the apparatus and methods of the invention and should not be interpreted as limiting the scope of the invention in any way.

Referring first to FIG. 5A, there is shown the exemplar phrenic waveform signal 72, which has been modified to exclude the negative half of the transmitted signal. The signal 72 comprises only two segments, the initial segment 74 and the spike segment 76.

Referring now to FIG. 5B, there is shown the exemplar phrenic waveform signal 78 that has been frequency modulated at 500 Hz. The signal 78 includes the same two segments, the initial segment 80 and the spike segment 82.

### Example 2

A study was performed to locate the phrenic nerve in the neck and stimulate the diaphragm. A neurocomputer embodying features of the invention was used to store and process captured waveform signals and generate waveform signals operative in controlling the diaphragm. A 0.58 kg rat was anesthetized; the neck, the back of the neck and chest were shaved. A tracheotomy was performed and the rat was intubated using a 14 g catheter. An incision was made at the back of the neck to locate the spine. A dremmel tool was used to perform a laminectomy and sever the spinal cord at C-2, C-3. Diaphragm and intercostal movement stopped.

The tracheotomy incision was extended to locate the right phrenic in the neck. The isoflurane was then reduced from 1 to 0.25% and the oxygen flow was then reduced to 0.3 L/min.

A hook probe was attached to the right phrenic nerve in the neck. The red (signal) lead was attached to the hook probe and the black (ground) lead was attached to an exposed muscle in the neck.

Using waveform signals generated by the neurocomputer, stimulation began at 2:35 pm with strong diaphragm movement, and stopped at 9:35 pm. Throughout the seven hours, the rat was “breathing” using the input signal. As reflected in Table I, vital signs were within normal limits.

TABLE I

| | Heart | | | | 02 | |
|---|---|---|---|---|---|---|
| | Rate | Blood | | | Level | Degree of |
| Time | BPM | Pressure | SPO2 | TEMP | L/min | Movement |
| 2:37 pm | 246 | | 98 | 96.4 | 0.3 | Strong |
| 2:45 pm | 246 | | 93 | 95.9 | 0.3 | Strong |
| 2:55 pm | 212 | 84/54 | 93 | 94.8 | 0.3 | Strong |
| 3:05 pm | 248 | 94/61 | 91 | 94.3 | 0.3 | Strong |
| 3:35 pm | 238 | 57/27 | 89 | 94.5 | 0.3 | Strong |
| 4:15 pm | 212 | 77/45 | 94 | 94.8 | 0.3 | Strong |
| 4:35 pm 2 hrs | 216 | 69/49 | 94 | 95 | 0.3 | Strong |
| 4:55 pm | 230 | 86/57 | 94 | 95.7 | 0.3 | Strong |
| 5:15 pm | 212 | 89/47 | 93 | 95.4 | 0.3 | Strong |
| 5:40 pm 3 hrs | 220 | 74/40 | 95 | 93.6 | 0.3 | Strong |
| 6:00 pm | 204 | 69/44 | 95 | 93.6 | 0.3 | Strong |
| 6:20 pm | 192 | 67/40 | 96 | 91 | 0.3 | Strong |
| 6:30 pm 4 hrs | 192 | 64/34 | 100 | 91.8 | 0.3 | Strong |
| 6:50 pm | 218 | 55/24 | 96 | 94.3 | 0.3 | Strong |
| 7:10 pm | 208 | 69/35 | 98 | 93.9 | 0.3 | Strong |
| | | SQ fluids | | | | |
| | | to rat | | | | |
| 7:30 pm 5 hrs | 210 | 74/40 | 98 | 94.5 | 0.3 | Strong |
| 7:50 pm | 208 | 76/42 | 98 | 95.5 | 0.3 | Strong |
| 8:10 pm | 220 | 74/40 | 99 | 94.8 | 0.3 | Strong |
| 8:30 pm 6 hrs | 226 | 72/40 | 98 | 95 | 0.3 | Strong |
| 8:50 pm | 222 | 71/39 | 97 | 95.5 | 0.3 | Strong |
| 9:10 pm | 224 | 72/40 | 96 | 96.4 | 0.3 | Strong |
| 9:20 pm | 200 | 77/53 | 94 | 96.3 | 0.3 | Strong |
| 9:35 pm 7 hrs | 218 | Signal | 87 | 96.3 | 0.3 | Strong |
| | | stopped | | | | |

### Example 3

A study was performed to locate the phrenic nerve in the neck and stimulate the diaphragm. A neurocomputer embodying features of the invention was used to store and process captured waveform signals, and generate waveform signals operative in controlling the diaphragm. A 0.74 kg rat was anesthetized; the neck, the back of the neck, and chest were shaved, a tracheotomy was performed. The rat was intubated using a 14 g catheter.

An incision was made at the back of the neck to locate the spine. A dremmel tool was used to perform a laminectomy and sever the spinal cord at C-2, C-3. Diaphragm and intercostals movement stopped.

The tracheotomy incision was extended to locate the right phrenic in the neck. The isoflurane was then reduced from 1 to 0.25% and the oxygen flow was reduced to 0.3 L/min.

A hook probe was attached to the right phrenic nerve in the neck. The red (signal) lead was attached to the hook probe and the black (ground) lead was attached to an exposed muscle in the neck. Using a waveform generated by the neurocomputer that corresponded to a recorded waveform signal stored in the neurocomputer, stimulation began at 3:50 pm with strong diaphragm movement. At 4:05 pm, the intercostals muscles began moving on their own again. Stimulation was stopped and another attempt was made to completely sever the spinal cord. Intercostal movement stopped. The probe was reattached to the right phrenic but no movement resulted when stimulated. The left phrenic was then located and the hook probe was attached.

Using waveform signals generated by the neurocomputer, stimulation started at 4:30 pm with good strong diaphragm movement and continued until 7:30 pm when the study was ended. As reflected in Table II, vital signs were within the normal limits throughout the three hours that the rat was “breathing”.

TABLE II

| | Heart Rate | | | 02 Level | Degree of |
|---|---|---|---|---|---|
| Time | BPM | SPO2 | TEMP | L/min | Movement |
| 3:50 pm | 3.32 | 98 | 96.3 | 0.3 | Strong |
| 4:00 pm | 284 | 92 | 93.6 | 0.3 | Strong |
| 4:40 pm | 266 | 99 | 97 | 0.3 | Strong |
| 4:50 pm | 260 | 100 | 97.5 | 0.3 | Strong |
| 5:00 pm | 254 | 99 | 97.7 | 0.3 | Strong |
| 5:10 pm | 250 | 99 | 98.2 | 0.3 | Strong |
| 5:20 pm | 246 | 99 | 98.1 | 0.3 | Strong |
| 5:30 pm 1 hr | 250 | 99 | 98.2 | 0.3 | Strong |
| 5:40 pm | 236 | 98 | 98.1 | 0.3 | Strong |
| 5:50 pm | 242 | 100 | 97.7 | 0.25 | Strong |
| 6:00 pm | 242 | 98 | 97.5 | 0.25 | Strong |
| 6:20 pm | 234 | 97 | 97.9 | 0.25 | Strong |
| 6:30 pm 2 hrs | 238 | 98 | 97.5 | 0.25 | Strong |
| 6:40 pm | 242 | 98 | 97.5 | 0.25 | Strong |
| 6:50 pm | 240 | 98 | 97.5 | 0.25 | Strong |
| 7:00 pm | 232 | | 97.7 | 0.25 | Strong |
| 7:10 pm | 234 | 100 | 97.2 | 0.25 | Strong |
| 7:20 pm | 232 | 100 | 97 | 0.25 | Strong |
| 7:30 pm 3 hrs | 238 | 99 | 97 | 0.25 | Strong |

As will be appreciated by one having ordinary skill in the art, the neurocomputer and neurocode system for recording, storing, analyzing, processing and transmitting waveform signals described above provides numerous advantages. Among the advantages are the provision of a neurocomputer that is adapted to:
Receive and process neurosignals (or waveform signals) generated in a body in “real-time”
Generate waveform signals that substantially correspond to waveform signals that are generated in the body and are operative in the control of body organ function
Store categorized waveform signals that are generated in the body and waveform signals that are generated by the processing means of the neurocomputer
Modify captured waveform signals and segments thereof
Generate baseline waveform signals from captured waveform signals
Compare captured waveform signals to baseline signals and generate a waveform signal based on the comparison

A further advantage is the provision of a neurocomputer that can be readily incorporated into an integrated system having means for capturing waveform signals from a subject and communicating the signals to the neurocomputer and means for transmitting (or delivering) generated waveform signals to the subject.

The neurocomputer and neurocode system of the invention can also be employed in numerous applications to control one or more body functions. Among the envisioned applications are the treatment or assessment of sleep apnea, respiratory distress, asthma, acute low blood pressure, abnormal heart beat, paralysis, spinal chord injuries, acid reflux, obesity, erectile dysfunction, a stroke, tension headaches, a weakened immune system, irritable bowel syndrome, low sperm count, sexual unresponsiveness, muscle cramps, insomnia, incontinence, constipation, nausea, spasticity, dry eyes syndrome, dry mouth syndrome, depression, epilepsy, low levels of growth hormone and insulin, abnormal levels of thyroid hormone, melatonin, adrenocorticotropic hormone, ADH, parathyroid hormone, epinephrine, glucagon and sex hormones, pain block and/or abatement, physical therapy and deep tissue injury.

Specific examples of organ and/or system control (and medical applications associated therewith) using the neurocode systems of the invention described herein are disclosed in co-pending U.S. patent application Ser. Nos. 10/781,078, filed Feb. 18, 2004, Ser. No. 10/847,738, filed May 17, 2004, Ser. No. 10/871,928, filed Jun. 18, 2004, Ser. No. 10/889,407, filed Jul. 12, 2004, Ser. No. 10/897,700, filed Jul. 23, 2004, Ser. No. 10/945,463, filed Sep. 20, 2004, Ser. No. 10/982,093, filed Nov. 4, 2004, Ser. No. 11/125,480, filed May 9, 2005, Ser. No. 11/129,264, filed May 9, 2005, Ser. No. 11/134,767, filed May 20, 2005, Ser. No. 60/592,751, filed Jul. 30, 2004, Ser. No. 60/601,233, filed Aug. 13, 2004, Ser. No. 60/602,435, filed Aug. 18, 2004, Ser. No. 60/604,279, filed Aug. 24, 2004, and Ser. No. 60/604,669, filed Aug. 25, 2004, all of which are incorporated herein in their entirety.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

1. A neurocomputer for regulating body organ function, comprising a means for receiving waveform signals, a storage medium to store received waveform signals, means for processing said stored waveform signals and means for generating at least one waveform signal that is operative in the control of body organ function.

2. The neurocomputer of claim 1, wherein said means for receiving waveform signals is adapted to receive signals having a rate of at least approximately 10,000 S/sec.

3. The neurocomputer of claim 2, wherein said means for receiving waveform signals is adapted to receive signals having a sample rate of approximately 1 MS/sec.

4. The neurocomputer of claim 1, wherein said storage medium stores received waveform signals categorized by body organ function.

5. The neurocomputer of claim 1, wherein said means for processing stored waveform signals retrieves a selected waveform signal from said storage medium.

6. The neurocomputer of claim 5, wherein said means for processing stored waveform signals modifies said stored waveform signal.

7. The neurocomputer of claim 6, wherein said means for processing stored waveform signals modifies said stored waveform signal by adjusting a characteristic selected from the group consisting of frequency, voltage and pacing.

8. The neurocomputer of claim 6, wherein said means for processing stored waveform signals frequency modulates said stored waveform signal.

9. The neurocomputer of claim 8, wherein said means for processing stored waveform signals frequency modulates said stored waveform signal to approximately 500 Hz.

10. The neurocomputer of claim 6, wherein said means for processing stored waveform signals modifies a segment of said stored waveform signal by performing an operation selected from the group consisting of copying, cutting, pasting, deleting, cropping, building, appending and inserting.

11. The neurocomputer of claim 6, wherein said means for processing stored waveform signals comprises generating a baseline signal from said stored waveform signals.

12. The neurocomputer of claim 1, wherein said means for generating a waveform signal is adapted to provide signals having a rate of approximately 5 Mbps.

13. The neurocomputer of claim 12, wherein said means for generating a waveform signal is adapted to provide signals having a rate of approximately 1 Mbps.

14. A computerized system for recording, storing, processing and transmitting waveform signals to regulate body organ function, comprising:
a first sensor for capturing a first waveform signal that is generated in a subject's body and is operative in the regulation of body organ function;
a neurocomputer adapted to receive said captured waveform signal, store said captured waveform signal, process said stored waveform signal and generate a second waveform signal that substantially corresponds to said first waveform signal and is recognizable by at least one body organ as a modulation signal; and
a transmitter for delivering said second waveform signal to said subject's body.

15. The system of claim 14, wherein said first waveform signal captured by said sensor is converted from analog form to digital form.

16. The system of claim 14, wherein said first sensor is adapted to provide direct connection to a nerve in said subject.

17. The system of claim 14, wherein said first sensor is adapted to provide indirect communication with said subject's body.

18. The system of claim 14, wherein said neurocomputer modifies said stored waveform signal by adjusting a characteristic selected from the group consisting of frequency, voltage and pacing.

19. The system of claim 18, wherein said processor frequency modulates said stored waveform signal.

20. The system of claim 19, wherein said processor frequency modulates said stored waveform signal to approximately 500 Hz.

21. The system of claim 14, wherein said first sensor comprises a high speed sensor.

22. The system of claim 21, wherein said first sensor has a sample rate of at least approximately 10,000 S/sec.

23. The system of claim 22, wherein said first sensor has a sample rate of approximately 1 MS/sec.

24. The system of claim 14, wherein said transmitter is adapted to provide direct connection to a nerve in said subject.

25. The system of claim 14, wherein said transmitter is adapted to provide indirect communication with said subject's body.

26. The system of claim 25, wherein said transmitter provides connection to said nerve using energy selected from the group consisting of magnetic, electromagnetic, ultrasonic, sonic, seismic and broadband.

27. The system of claim 14, further comprising a second sensor.

28. The system of claim 27, wherein said second sensor comprises a low speed sensor.

29. The system of claim 28, wherein said second sensor is selected from the group consisting of a respirator, a pneumotach, a pulse rate monitor, an airflow monitor, a vitals monitor, a temperature sensor, a motion sensors and a pressure sensor.
